(19) United States
(12) Patent Application Publication
(10) Pub. No.: US 2023/0056172 A1
(43) Pub. Date: Feb. 23, 2023

(54) DIAGNOSIS SUPPORT APPARATUS AND METHOD FOR SUPPORTING DIAGNOSIS

(71) Applicant: CANON MEDICAL SYSTEMS CORPORATION, Otawara-shi (JP)

(72) Inventors: Yoshifumi YAMAGATA, Sakura (JP); Kouji OTA, Nasushiobara (JP); Seito IGARASHI, Nasushiobara (JP); Koji TAKEI, Nasushiobara (JP); Hidetoshi ISHIGAMI, Otawara (JP); Yohei KAMINAGA, Otawara (JP)

(73) Assignee: CANON MEDICAL SYSTEMS CORPORATION, Otawara-shi (JP)

(21) Appl. No.: 17/819,807

(22) Filed: Aug. 15, 2022

(30) Foreign Application Priority Data
Aug. 18, 2021 (JP) 2021-133432

(51) Int. Cl. A61B 6/00; G16H 50/20

(52) U.S. Cl. CPC A61B 6/5217; G16H 50/20; G16H 50/70

(57) ABSTRACT

The diagnosis support apparatus according to any of embodiments includes processing circuitry. The processing circuitry is configured to acquire external force data regarding external force applied to a subject. The processing circuitry is configured to generate diagnosis support data for supporting diagnosis practice to the subject based on the acquired external force data. The processing circuitry is configured to control output of the generated diagnosis support data.

### CROSS-REFERENCE TO RELATED APPLICATION

This application is based upon and claims the benefit of priority from Japanese Patent Application No. 2021-133432, filed on Aug. 18, 2021, the entire contents of which are incorporated herein by reference.

### FIELD

Any of embodiments disclosed in specification and drawings relates to a diagnosis support apparatus and a method for supporting diagnosis.

### BACKGROUND

Emergency diagnostic treatment for traffic injuries has two issues: early medical treatment and accurate diagnosis. The early medical treatment requires medical treatment to be started as soon as possible after an accident occurs, which can be realized by a conventional emergency report system. On the other hand, it is extremely difficult to quickly and accurately grasp the injury information of the injured person who is brought to the hospital. If the patient is unconscious, the doctor cannot ask the patient how the injury is made or where the patient may feel the pain. In such case, except visible injuries such as incised blood, fractures, and dislocations, the injuries are presumed based on basic medical data such as pulse, blood pressure, heart rate, electrocardiogram, and brain waves.

Even if the injured person is conscious, in the case of visceral injury or cranial nerve injury for example, the person may not feel any abnormalities such as pain immediately after the injury. In such case, unless the patient declaring a bruise, detailed examinations such as computed tomography (CT) apparatuses and magnetic resonance imaging (MRI) apparatuses may not be performed, which may delay medical treatment for significant hidden injuries. In view of such problems, an emergency report system that enables accurate diagnosis by detecting and reporting the load on the occupant due to a collision accident is disclosed.

In addition, for high-energy trauma (e.g., a fall from a high place, a car accident occurred at a certain speed or higher, etc.), the diagnosis is advanced with reference to the trauma initial medical treatment guideline. In the second stage (secondary survey) of the three-stage interpretation of the Japan Advanced Trauma Evaluation Guideline, diagnosis is made by systematic body inspection of damages to the subject.

### BRIEF DESCRIPTION OF THE DRAWINGS

FIG. 1 is a schematic diagram showing a configuration of a diagnosis support apparatus according to the first embodiment.

FIG. 2 is a schematic diagram showing a configuration of the diagnosis support system provided with the diagnosis support apparatus according to the first embodiment.

FIG. 3 is a block diagram showing an example of functions of the diagnosis support apparatus according to the first embodiment.

FIG. 4 is a diagram showing the method for supporting the diagnosis with the diagnosis support apparatus according to the first embodiment as a flowchart.

Each of FIGS. 5A and 5B is a diagram showing the first display example of superimposed data in the diagnosis support apparatus according to the first embodiment.

FIG. 6 is a diagram showing the second display example of superimposed data in the diagnosis support apparatus according to the first embodiment.

FIG. 7 is a diagram showing the third display example of superimposed data in the diagnosis support apparatus according to the first embodiment.

FIG. 8 is a diagram showing the fourth display example of superimposed data in the diagnosis support apparatus according to the first embodiment.

FIG. 9 is a diagram showing the fifth display example of superimposed data in the diagnosis support apparatus according to the first embodiment.

Each of FIGS. 10A and 10B is a diagram showing the sixth display example of superimposed data in the diagnosis support apparatus according to the first embodiment.

FIG. 11 is a schematic diagram showing a configuration of the diagnosis support system provided with the diagnosis support apparatus according to the second embodiment.

FIG. 12 is an explanatory diagram showing an example of a data flow at the time of learning in the diagnosis support apparatus according to the second embodiment.

FIG. 13 is an explanatory diagram showing an example of a data flow during operation in the diagnosis support apparatus according to the second embodiment.

FIG. 14 is an explanatory diagram showing an example of a data flow at the time of learning in the diagnosis support apparatus according to the third embodiment.

FIG. 15 is an explanatory diagram showing an example of a data flow during operation in the diagnosis support apparatus according to the third embodiment.

FIG. 16 is an explanatory diagram showing an example of a data flow at the time of learning in the diagnosis support apparatus according to the fourth embodiment.

FIG. 17 is an explanatory diagram showing an example of a data flow during operation in the diagnosis support apparatus according to the fourth embodiment.

FIG. 18 is a diagram showing an example of displaying injured region data in the diagnosis support apparatus according to the fourth embodiment.

FIG. 19 is an explanatory diagram showing an example of a data flow at the time of learning in the diagnosis support apparatus according to the fifth embodiment.

FIG. 20 is an explanatory diagram showing an example of a data flow during operation in the diagnosis support apparatus according to the fifth embodiment.

FIG. 21 is an explanatory diagram showing an example of a data flow at the time of learning in the diagnosis support apparatus according to the seventh embodiment.

FIG. 22 is an explanatory diagram showing an example of a data flow during operation in the diagnosis support apparatus according to the seventh embodiment.

### DETAILED DESCRIPTION

A diagnosis support apparatus and a method for supporting diagnosis according to any of embodiments will be described with reference to the accompanying drawings.

The diagnosis support apparatus according to any of embodiments includes processing circuitry. The processing circuitry is configured to acquire external force data regarding external force applied to a subject. The processing circuitry is configured to generate diagnosis support data for supporting diagnosis practice to the subject based on the acquired external force data. The processing circuitry is configured to control output of the generated diagnosis support data.

### First Embodiment

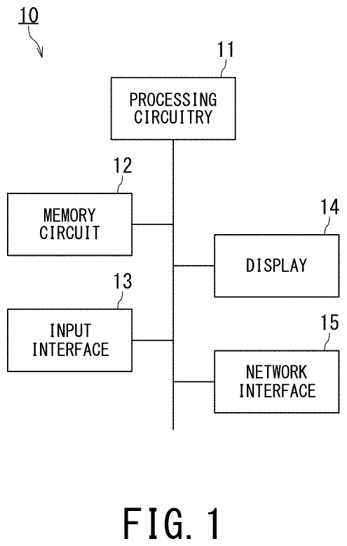

FIG. 1 is a schematic diagram showing a configuration of the diagnosis support apparatus according to the first embodiment.

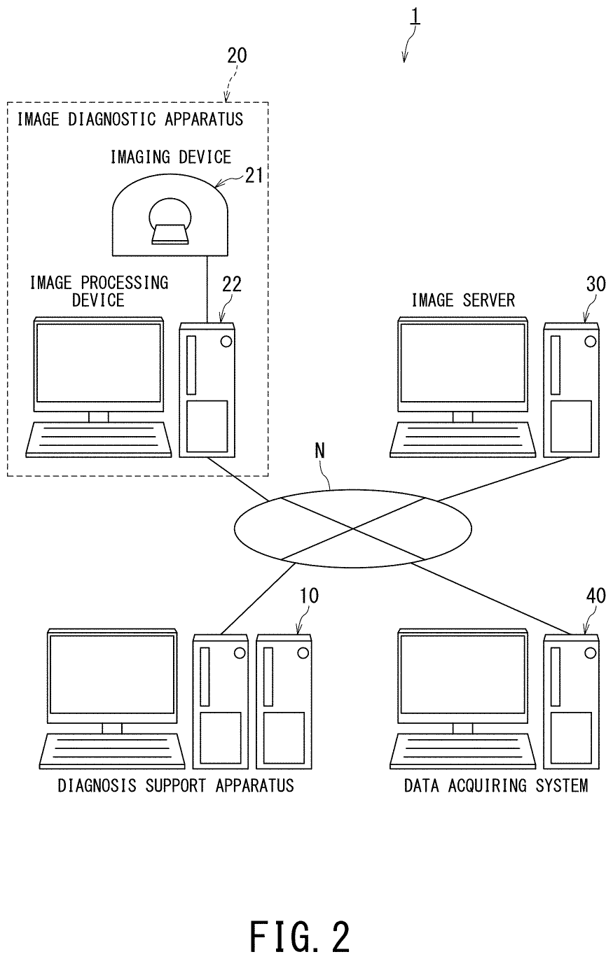

FIG. 1 shows a diagnosis support apparatus 10 according to the first embodiment. The diagnosis support apparatus 10 is an image diagnostic apparatus, a data server, a workstation, an image interpretation terminal, or the like, and is provided in a medical imaging system connected via a network N (shown in FIG. 2). The diagnosis support apparatus 10 may be an offline device.

The diagnosis support apparatus 10 includes processing circuitry 11, a memory circuit 12, an input interface 13, a display 14, and a network interface 15.

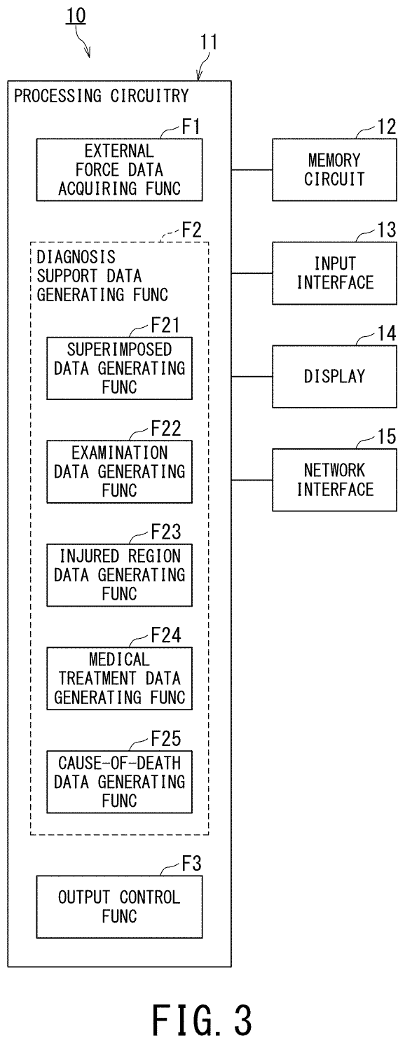

The processing circuitry 11 controls the operation of the diagnosis support apparatus 10 according to the input operation received from the operator via the input interface 13. For example, the processing circuitry 11 is realized by a processor. Functions of the processing circuitry 11 will be described later with reference to FIG. 3.

The memory circuit 12 is composed of a semiconductor memory element such as a random access memory (RAM) and a flash memory, a hard disk, an optical disk, and the like. The memory circuit 12 may be configured by a portable medium such as a universal serial bus (USB) memory and a digital video disk (DVD). The memory circuit 12 stores various processing programs (including an operating system (OS) and the like in addition to the application program) used in the processing circuitry 11 and data necessary for executing the program. Further, the OS may include a graphical user interface (GUI) which makes extensive use of graphics for displaying data on the display 14 to the operator and allows basic operations to be performed by the input interface 13. The memory circuit 12 is an example of a memory unit.

The input interface 13 includes an input device which can be operated by an operator and an input circuit which inputs a signal from the input device. The input device is realized by a trackball, a switch, a mouse, a keyboard, a touch pad where input operation is made by touching an operation surface, a touch screen where a display screen and a touch pad is integrated, a contactless input device using an optical sensor, a voice input device, and the like. When the input device is operated by the operator, the input circuit generates a signal corresponding to the operation and outputs the signal to the processing circuitry 11. The diagnosis support apparatus 10 may include a touch panel in which the input device integrated with the display 14. Further, the input device is not limited to the one having physical operating components such as a mouse and a keyboard. For example, the input circuit may receive an electric signal corresponding to the input operation from an external input device provided separately from the diagnosis support apparatus 10, and output the electric signal to the processing circuitry 11. The input interface 13 is an example of an input unit.

The display 14 is a display device such as a liquid crystal display panel, a plasma display panel, and an organic electro luminescence (EL) panel. The display 14 is connected to the processing circuitry 11 and displays various data and images generated under the control of the processing circuitry 11. The display 14 is an example of an output unit. Further, the diagnosis support apparatus 10 may be provided with a speaker (not shown) or the like as another output unit. The speaker is a device that converts an electric signal which represents sound (hereinafter referred to as “acoustic signal”) into a physical sound, that is, vibration of air.

The network interface 15 is composed of connectors which meet the parallel connection specifications and the serial connection specifications. The network interface 15 has a function of performing communication control according to each standard and connecting the network N (shown in FIG. 2) through a telephone line. Thereby, the diagnosis support apparatus 10 can be connected to the network. The network interface 15 is an example of a communication unit.

Subsequently, the system to which the diagnosis support apparatus 10 is applied will be described.

FIG. 2 is a schematic diagram showing a configuration of the diagnosis support system provided with the diagnosis support apparatus 10.

FIG. 2 shows a diagnosis support system 1 provided with the diagnosis support apparatus 10. The diagnosis support system 1 includes the diagnosis support apparatus 10 shown in FIG. 1, one (or more) image diagnostic apparatus 20, one (or more) image server 30, and one (or more) data acquiring system 40. The diagnosis support apparatus 10, the image diagnostic apparatus 20, the image server 30, and the data acquiring system 40 are connected via the network N that enables communication with each other. An electrical connection or the like via an electronic network can be applied to this connection. Here, the electronic network refers to a general information communication network using telecommunications technology, such as a wireless/wired hospital backbone local area network (LAN), an Internet network, a telephone communication line network, an optical fiber communication network, a cable communication network, a satellite communication network, and the like.

The image diagnostic apparatus 20 is an apparatus for generating a medical image. The image diagnostic apparatus 20 includes an X-ray diagnostic apparatus, an X-ray computed tomography (CT) apparatus, an magnetic resonance imaging (MRI) apparatus, a nuclear medicine diagnostic apparatus, an ultrasonic diagnostic apparatus, and the like. The image diagnostic apparatus 20 includes an imaging device 21 and an image processing device 22.

The imaging device 21 acquires data as the basis of medical image data. The image processing device 22, which has a general configuration of a computer, controls the operation of the imaging device 21, acquires data from the imaging device 21, and process the data to generate medical image data. When the image diagnostic apparatus 20 is an X-ray diagnostic apparatus, the imaging device 21 includes an X-ray tube, an X-ray detector, and the like. When the image diagnostic apparatus 20 is an X-ray CT apparatus, the MRI apparatus, or the nuclear medicine diagnostic apparatus, the imaging device 21 is a so-called gantry. When the image diagnostic apparatus 20 is an ultrasonic diagnostic apparatus, the imaging device 21 is a so-called ultrasonic probe.

The image processing device 22 includes a general configuration of a computer. For example, the image processing device 22 includes processing circuitry, a memory circuit, an input interface, a display, and a network interface (not shown).

The image server 30 includes a general configuration of a computer. For example, the image server 30 includes processing circuitry, a memory circuit, an input interface, a display, and a network interface (all not shown). The configuration of the processing circuitry, the memory circuit, the input interface, the display, and the network interface of the image server 30 is the same as that of processing circuitry 11, the memory circuit 12, the input interface 13, the display 14, and the network interface 15 shown in FIG. 1 respectively, so the description thereof will be omitted.

The image server 30 is, for example, a digital imaging and communications in medicine (DICOM) server, and is connected to a device, such as the image diagnostic apparatus 20, that can transmit and receive data via a network N. The image server 30 manages medical image data such as CT image data generated by the image diagnostic apparatus 20 as a DICOM file.

The data acquiring system 40 acquires internal and external data of the vehicle, and/or person's fall detection data. The internal and external data of the vehicle is collected by sensors mounted on vehicles such as general vehicles, autonomous vehicles, and connected cars which came across a traffic accident. The person's fall detection data is collected by smart homes, watching systems, and the like.

FIG. 3 is a block diagram showing an example of functions of the diagnosis support apparatus 10.

As shown in FIG. 3, the processing circuitry 11 reads out and executes a computer program stored in the memory circuit 12 or directly incorporated in the processing circuitry 11, thereby realizing an external force data acquiring function F1, a diagnosis support data generating function F2, and an output control function F3. Hereinafter, a case where the functions F1 to F3 function as software by execution of the computer program will be described as an example. However, all or part of the functions F1 to F3 may be realized by a circuit such as an ASIC. Further, all or a part of the functions F1 to F3 may be realized by the image processing device 22 or the image server 30 of the image diagnostic apparatus 20.

The external force data acquiring function F1 includes a function of acquiring external force data regarding the external force applied to the subject. Here, the external force data includes external force indicating data for presenting the external force applied to the subject and medical image data (hereinafter, simply referred to as “medical image data”) of the subject to which the external force is applied. First, the external force indicating data will be described. The subject to which an external force is applied is a target for diagnosis.

For high-energy trauma (e.g., a fall from a high place, a car accident occurred at a certain speed or higher, etc.), the diagnosis is advanced with reference to the trauma initial medical treatment guidelines. In the second stage (secondary survey) of the three-stage interpretation of the Japan Advanced Trauma Evaluation Guideline, diagnosis is made by systematic body inspection of damages. Then, based on the injury mechanism (cause and circumstances leading to the injury), the external force indicating data, which includes the input position, the input direction, and the strength (magnitude) of the energy (the external force) being applied to the subject, is presumed, and the damage search is performed. However, when the subject has difficulty stating the injury mechanism, there can be pit-falls. Therefore, the external force data acquiring function F1 acquires internal and external data of the vehicle, and/or the person's fall detection data from the data acquiring system 40, and acquires the external force indicating data based on internal and external data of the vehicle, and/or the person's fall detection data.

First, the external force data acquiring function F1 can acquire, as image data, internal and external data of the vehicle at the time of a traffic accident from the data acquiring system 40 using an event data recorder (EDR) for traffic accident analysis. The technical requirements of EDR include the physique and position classification of occupants in the vehicle. For example, the image data of each type of the vehicle having an accident in an undamaged state may be stored in advance in the storage circuit 12. Also, the image data of the vehicle having the accident and the external force indicating data of the occupant in the vehicle having the accident (that is, the input position, the input direction, and the strength (magnitude) of the external force) may be associated and stored in the memory circuit 12 in advance. The external force data acquiring function F1 then compares the image data of the vehicle having the accident with the pre-stored image data of the same type of vehicle in an undamaged state, and acquires the external force indicating data of the occupant in the vehicle having the accident.

Second, the external force data acquiring function F1 can acquire image data showing the posture of a fall detected by an AI camera (fall detection camera) installed indoors as the person's fall detection data when an indoor fall accident happens from the data acquiring system 40. For example, the image data of each posture of the fell person during the fall may be stored in the memory circuit 12 in advance. Also, such image data and the external force indicating data of the fell person (that is, the input position, the input direction, and the strength (magnitude) of the external force) may be associated and stored in the memory circuit 12 in advance. The external force data acquiring function F1 then compares the image data of the fell person with the pre-stored image data of each posture during the fall, and acquires the external force indicating data for the fallen person. The external force data acquiring function F1 may acquire the person's fall detection data of an indoor fall accident from a mobile terminal or a wearable device.

Further, the external force data acquiring function F1 acquires, as the external force data, medical image data (e.g., CT image data, MRI image data, or the like) of a subject to which an external force is applied.

The diagnosis support data generating function F2 includes a function of generating diagnosis support data that supports diagnosis practice to a subject based on the external force data (at least one of the external force indicating data and the medical image data) acquired by the external force data acquiring function F1. The diagnosis support data generating function F2 includes a superimposed data generating function F21, an examination data generating function F22, an injured region data generating function F23, a medical treatment data generating function F24, and a cause-of-death data generating function F25.

The superimposed data generating function F21 includes a function of generating, as the diagnosis support data, superimposed data in which external force indicting data shown by symbols and/or characters is added to medical image data. Further, for example, the superimposed data generating function F21 generates superimposed data which is an acoustic signal representing the external force indicating data.

The examination data generating function F22 includes a function of generating, as the diagnosis support data, examination data relating to at least one of examination necessity and examination order (imaging plan, imaging range, etc.) based on the external force data or superimposed data acquired by the external force data acquiring function F1 (the third embodiment described later). The injured region data generating function F23 includes a function of generating, as the diagnosis support data, injured region data for identifying an injured region in a subject based on the external force data or superimposed data acquired by the external force data acquiring function F1 (the fourth embodiment described later). The medical treatment data generating function F24 includes a function of generating, as the diagnosis support data, medical treatment data representing the medical treatment plan (treatment plan, treatment or rehabilitation period) of the subject based on the external force data or superimposed data acquired by the external force data acquiring function F1 (the fifth embodiment described later). The CAUSE-OF-DEATH DATA GENERATING FUNCTION F25 includes a function of generating, as the diagnosis support data, cause-of-death data representing the cause of death of the subject based on the external force data or superimposed data acquired by the external force data acquiring function F1 (the sixth embodiment described later).

The output control function F3 includes a function of controlling the output of the diagnosis support data generated by the diagnosis support data generating function F2 to an output unit. Specifically, the output control function F3 controls the output of the diagnosis support data from the display 14 or from a speaker (not shown).

Subsequently, the method for supporting the diagnosis by the diagnosis support apparatus 10 will be described.

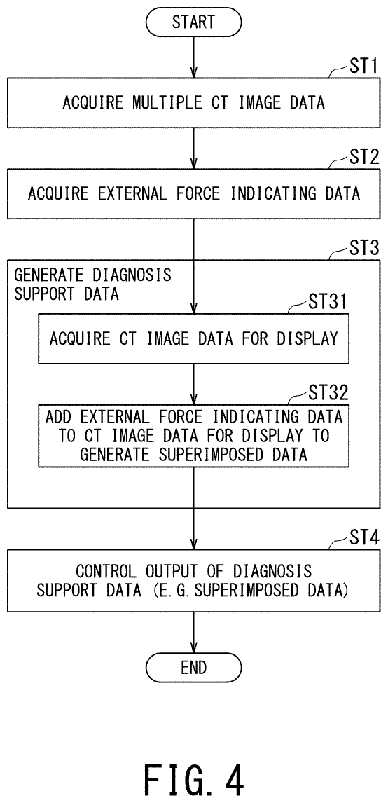

FIG. 4 is a diagram showing a method of processing a medical image file as a flowchart. In FIG. 4, the reference numeral “ST” with a number indicates each step of the flowchart. Here, in FIG. 4, the diagnosis support data will be described as the superimposed data in which external force indicating data, which is the external force data, is added to medical image data (e.g., CT image data), which is also external force data.

The external force data acquiring function F1 acquires CT image data of a subject, as external force data from the image processing device 22 of the image diagnostic apparatus 20 (step ST1). In step ST1, the external force data acquiring function F1 acquires CT image data including multiple CT images by whole-body imaging of the subject. The external force data acquiring function F1 acquires external force indicating data regarding the external force applied to the subject as external force data (step ST2).

The superimposed data generating function F21 of the diagnosis support data generating function F2 generates diagnosis support data, which supports diagnosis practice to the subject, based on the external force data (at least one of the medical image data and the external force indicating data) acquired in steps ST1 and ST2 (step ST3). In step ST3, the superimposed data generating function F21 acquires CT image data for display from the multiple CT image data acquired in step ST1 (step ST31). Then, the superimposed data generating function F21 generates, as the diagnosis support data, superimposed data in which external force indicating data as shown by symbols and/or characters is added to the CT image data, which is the medical image data, for display (step ST32).

The output control function F3 controls the output of the superimposed data generated in step ST32 from the display 14 (step ST4).

FIGS. 5A to 10B are diagrams showing first to sixth display examples of the superimposed data in step ST4, respectively.

FIG. 5A shows superimposed data in which external force indicating data is superimposed on two-dimensional (2D) CT image data. The external force indicating data is represented by an arrow. The position of the tip of the arrow refers to the input position (body surface) of the external force. The direction of the arrow refers to the input direction of the external force. The color within the arrow (corresponding to the gradation bar in FIG. 5A) represents the strength (absolute value) of the external force. The length of the arrow represents the strength of the external force (relative value compared with strength towards other input positions). The color consists of hue, saturation, and lightness, and at least one of which within the arrow is used to represent the difference in the absolute value of the strength of the external force. In this way, since the tip of the arrow is shown near the body surface of the subject, the external force indicating data showing the external force being applied to the subject is superimposed and displayed on the 2D CT image, which will not interfere with the damage search by the operator. On the display shown in FIG. 5A, it is also possible to change the tomographic position of the displayed superimposed data by following the operation via the input interface 13.

According to the display in FIG. 5A, by superimposing the external force indicating data (arrow), which represents the external force applied to the subject, on the 2D CT image, an operator such as a doctor can search for damage while visually confirming the external force indicating data as a guide.

FIG. 5B shows superimposed data in which external force indicating data is superimposed on three-dimensional (3D) CT image data. The external force indicating data is represented by an arrow. The meanings of the position of the tip of the arrow, the direction of the arrow, the color, and the length are the same as those shown in FIG. 5A. In this way, the external force indicating data indicating the external force applied to the subject can be superimposed and shown on the 3D CT image without interfering with the damage search by the operator. On the display shown in FIG. 5B, the projection direction of the displayed superimposed data can be changed by following the operation via the input interface 13.

According to the display in FIG. 5B, by superimposing the external force indicating data (arrow), which represents the external force applied to the subject, on the 3D CT image, the operator can perform the damage search while visually confirming the external force indicating data as a guide.

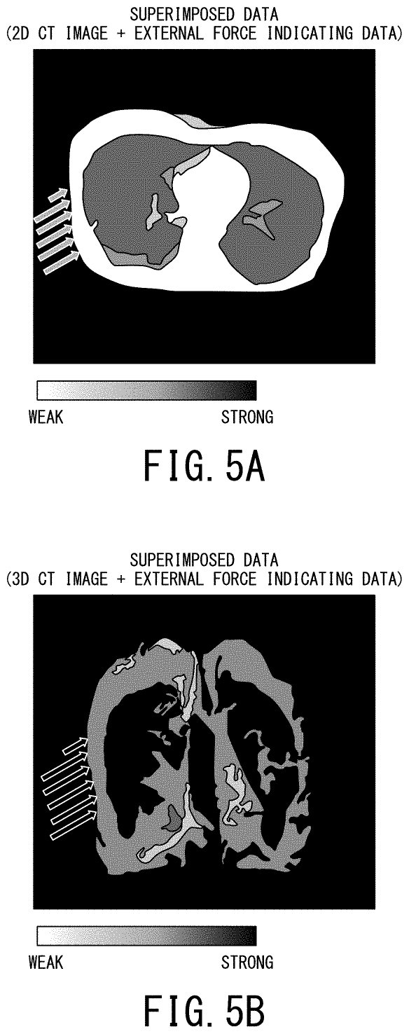

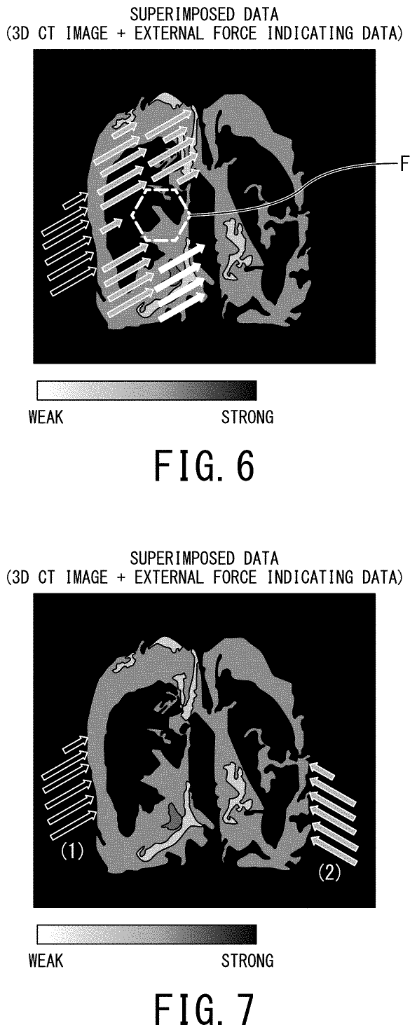

FIG. 6 shows superimposed data in which external force indicating data is superimposed on 3D CT image data. FIG. 6 shows an example in which the second external force indicating data is superimposed on the image of the subject included in the CT image in addition to the display of the first external force indication data shown in FIG. 5B. The second external force indicating data may be acquired from the external force propagation distribution as presumed by simulation or using an anatomical model of human body based on the first external force indicating data. In the case of the display shown in FIG. 6, it is conceivable that the second external force indicating data superimposed on the 3D CT image may interfere with the damage search by the operator. Therefore, the area where the operator is searching for damage is set as the non-display area F on the image of the CT image data. Then, the output control function F3 can non-display the external force indicating data in the non-display area F. Alternatively, the external force indicating data (that is, the arrow) can be set to be non-displayed in the area around the displayed mouse pointer (a circular or square area having a fixed length and centered on the mouse pointer). It is also possible to non-display the external force indicating data only near the center of the medical image data. On the display shown in FIG. 6, the projection direction of the superimposed data to be displayed can be changed by following the operation via the input interface 13. Further, although FIG. 6 is based on 3D CT image data, the same applies to the case based on 2D CT image data.

According to the display of FIG. 6, by superimposing the first and second external force indicating data (arrows) representing the external force applied to the subject, the operator can perform the damage search while visually confirming the first and second external force indicating data as a guide.

FIG. 7 shows superimposed data in which external force indicating data is superimposed on 3D CT image data. The external force indicating data is represented by an arrow and by characters like “(1)” and “(2)”. The meanings of the position of the arrow tip, the direction of the arrow, the color, and the length are the same as those shown in FIG. 5A. The display example in FIG. 7, different from the display example in FIG. 5B, shows a case when an external force is applied to the subject multiple times, for example, twice. That is, in FIG. 7, the case of multiple collisions and the like is illustrated, and the external force indicating data of individual collision is shown in a distinguishable manner. The character “(1)” refers to the first external force indicating data, and the character “(2)” refers to the second external force indicating data. On the display shown in FIG. 7, the projection direction of the superimposed data to be displayed can be changed by following the operation via the input interface 13. Further, although FIG. 7 is based on 3D CT image data, the same applies to the case based on 2D CT image data.

According to the display in FIG. 7, by superimposing multiple distinguishable external force indicating data (arrows), which represent multiple external forces applied to the subject multiple times, on the CT image, the operator can perform damage search while visually confirming multiple external force indicating data as a guide.

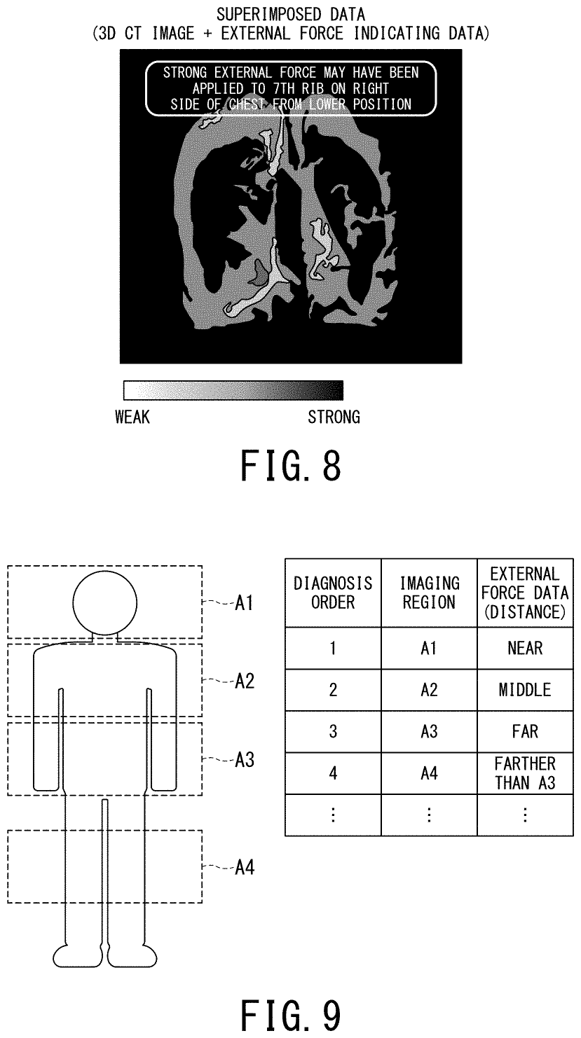

FIG. 8 shows superimposed data in which external force indicating data is superimposed on 3D CT image data. The external force indicating data is expressed by the frame around the characters and the character “STRONG EXTERNAL FORCE MAY HAVE BEEN APPLIED TO 7TH RIB ON RIGHT SIDE OF CHEST FROM LOWER POSITION”. On the display shown in FIG. 8, it is also possible to change the projection direction of the CT image data among the displayed superimposed data by following the operation via the input interface 13. Further, although FIG. 8 is based on 3D CT image data, the same applies to the case based on 2D CT image data.

According to the display of FIG. 8, by superimposing the external force indicating data (character string), which represents the external force applied to the subject, the operator can perform the damage search while visually confirming the external force indicating data as a guide.

The right side of FIG. 9 shows the diagnosis order (priority) to be performed by an operator such as a doctor among multiple imaging regions A1 to A4 as shown on the left side of the figure. The left side of FIG. 9 shows the positions of the multiple imaging regions A1 to A4. The superimposed data generating function F21 can acquire the diagnosis order (1 to 4) in addition to the above-mentioned superimposed data based on the external force indicating data. For example, the superimposed data generating function F21 can set the diagnosis priority of the imaging region A1 near the input position of the external force higher, and set the diagnosis priority of the imaging region A4 far from the input position of the external force lower. In the imaging region where important organs (e.g., brain and heart) are located, the diagnosis order can be decided including the weight factor in addition to the distance factor as mentioned above.

According to the display of FIG. 9, by providing the diagnosis order information, the operator can search for damage from the imaging region having a higher priority for life support.

FIG. 10A shows superimposed data in which external force indicating data is superimposed on 2D CT image data. The external force indicating data is represented by an arrow and a deformation line (broken line) showing the shape after deformation. The outer deformation line indicates deformation of the skin based on the external force indicating data. The inner deformation line shows the damage to the organ by the energy that reaches the organ (estimated value) based on the external force indicating data. On the display shown in FIG. 10, it is also possible to change the tomographic position of the displayed superimposed data by following the operation via the input interface 13. Further, although FIG. 10A is based on 2D CT image data, the same applies to the case based on 3D CT image data.

According to the display of FIG. 10A, by visualizing the deformation of the skin and organs based on the external force indicating data, the operator can search for damage while visually confirming the broken line after deformation as a guide. Further, as shown in FIG. 10B, the superimposed data generating function F21 may generate image data in which the CT image data is modified according to the deformation line.

As described above, according to the diagnosis support apparatus 10 in the first embodiment of the diagnosis support system 1, by using internal and external data of the vehicle or the person's fall detection data available from the data acquiring system 40 (shown in FIG. 2), the operator can search for damage while visually (or audibly) confirming the external force applied to the subject, which is more efficient as compared with the damage search that requires repeated confirmation of the injury mechanism with the subject. Further, according to the diagnosis support apparatus 10 in the first embodiment, a pitfall can be avoided since the explanation of the injury mechanism by the subject is not required. That is, according to the diagnosis support apparatus 10 in the first embodiment, superimposed data can be generated based on the external force indicating data of the subject based on internal and external data of the vehicle or the person's fall detection data available from the data acquiring system 40. Then, by outputting the superimposed data in the manner as shown in FIGS. 5A to 10B, it is possible to provide the operator who diagnoses the subject (including damage search) with effective diagnosis support data for the diagnosis.

### Second Embodiment

The method of generating superimposed data by the superimposed data generating function F21 shown in FIG. 3 is not limited to the above-mentioned method. For example, the superimposed data generating function F21 can generate superimposed data based on medical image data as an example of the external force data. This case will be described below.

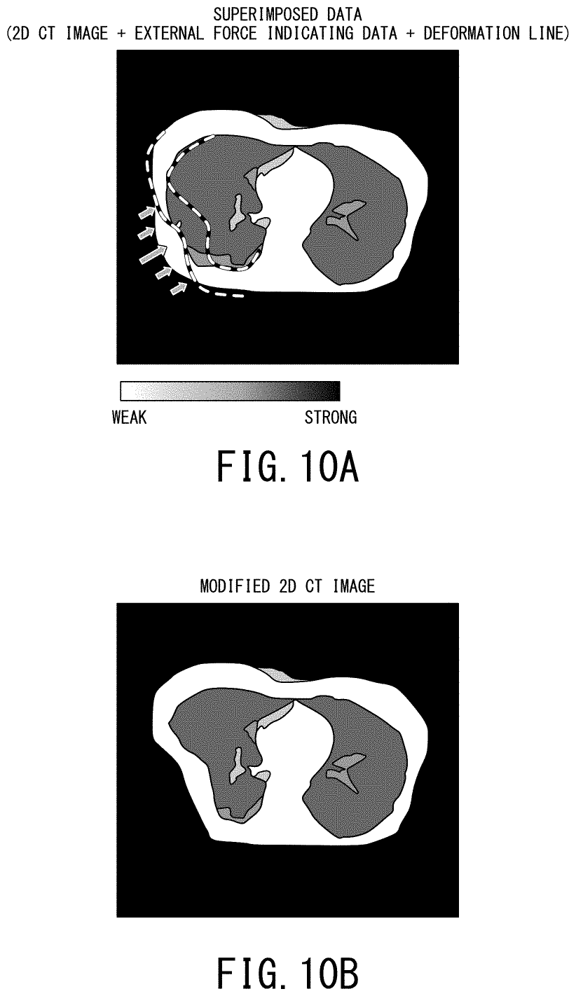

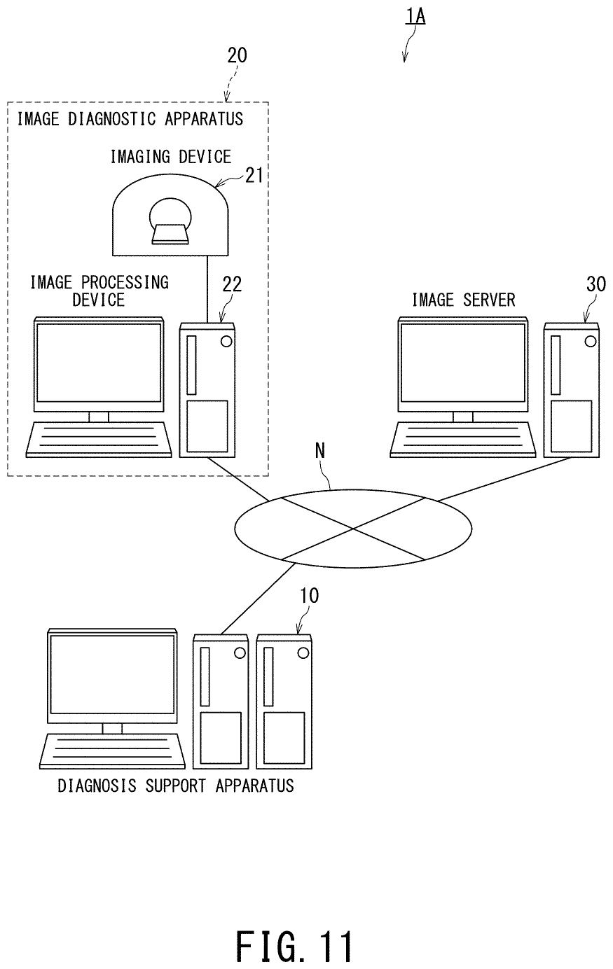

FIG. 11 is a schematic view showing a configuration of a diagnosis support system provided with the diagnosis support apparatus according to the second embodiment.

FIG. 11 shows a diagnosis support system 1A provided with the diagnosis support apparatus 10. The diagnosis support system 1A includes the diagnosis support apparatus 10 shown in FIG. 1, one or more image diagnostic apparatuses 20, and one or more image servers 30. The diagnosis support apparatus 10, the image diagnostic apparatus 20, and the image server 30 are connected via the network N that enables communication with each other. An electrical connection or the like via an electronic network can be applied to this connection.

The diagnosis support system 1A shown in FIG. 11 has a configuration in which the data acquiring system 40 is removed from the diagnosis support system 1 shown in FIG. 2. In FIG. 11, the same members as those shown in FIG. 2 are designated by the same reference numerals, and the description thereof will be omitted.

In the diagnosis support system 1A shown in FIG. 11, the superimposed data generating function F21 shown in FIG. 3 performs a process of generating superimposed data based on medical image data, for example, CT image data. For this process, for example, a look-up table (LUT) in which the CT image data and the superimposed data are associated with each other may be used. In addition, machine learning may be used for this process. Further, deep learning using a multi-layer neural network such as convolutional neural Network (CNN) or convolutional deep belief network (CDBN) may be used as the machine learning.

Here is an example showing that the superimposed data generating function F21 includes a neural network Nb and generates superimposed data based on the CT image data by using the deep learning. That is, the superimposed data generating function F21 inputs the CT image data of the subject into the trained model to generate the superimposed data of the subject.

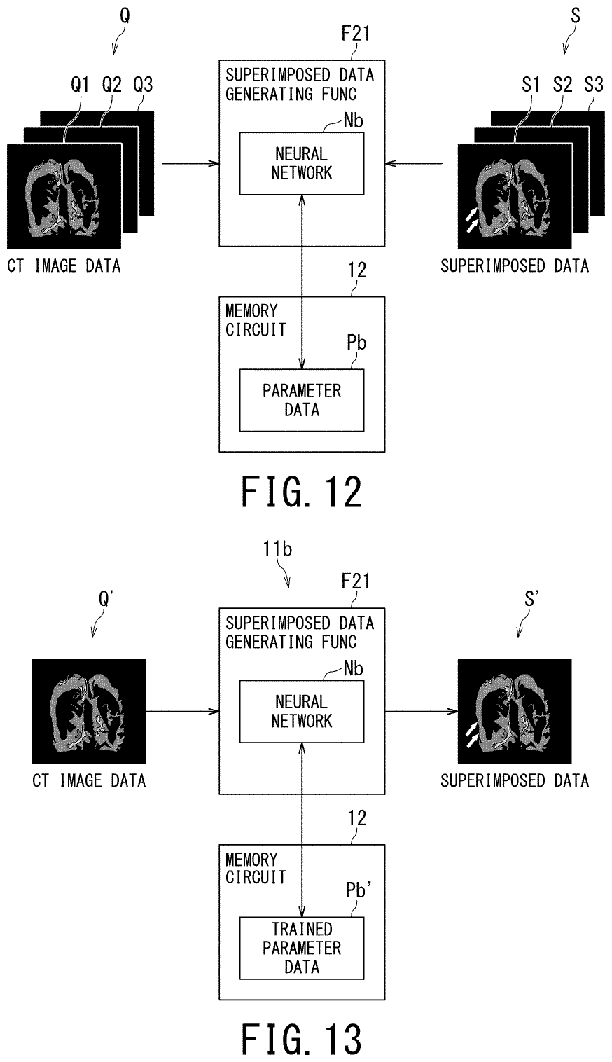

FIG. 12 is an explanatory diagram showing an example of the data flow at the time of learning.

The superimposed data generating function F21 sequentially updates the parameter data Pb by inputting a large number of training data and performing learning. The training data is composed of a combination of CT image data Q1, Q2, Q3, . . . , and superimposed data S1, S2, S3, . . . . The CT image data Q1, Q2, Q3 . . . constitutes a training input data group Q. The superimposed data S1, S2, S3, . . . constitutes the training output data group S. The superimposed data S1, S2, S3, . . . may correspond to the CT image data Q1, Q2, Q3, . . . , respectively.

The superimposed data generating function F21 updates the parameter data Pb such that, by the processing of the neural Nb, the CT image data Q1, Q2, Q3, . . . approaches the superimposed data S1, S2, S3, . . . each time training data is input, which is so-called learning. Generally, when the change rate of the parameter data Pb converges within the threshold value, it is determined that the learning is completed. Hereinafter, the parameter data Pb after learning is particularly referred to as learned parameter data Pb′.

It should be noted that the type of training input data and the type of input data during operation shown in FIG. 12 should be the same. For example, when the input data at the time of operation is the head CT image data of the subject, the training input data group Q at the time of learning should be the head CT image data.

Further, the “image data” includes raw data generated by the image diagnostic apparatus 20 (shown in FIG. 11). That is, the input data of the neural network Nb may be raw data before scan conversion.

FIG. 13 is an explanatory diagram showing an example of data flow during operation.

At the time of operation, the superimposed data generating function F21 inputs the CT image data Q′ of the subject which is the target of medical treatment, and outputs superimposed data S′ of the subject using the trained parameter data Pb′.

The neural network Nb and the trained parameter data Pb′ constitute the trained model 11b. The neural network Nb is stored in the memory circuit 12 in the form of a program. The trained parameter data Pb′ may be stored in the memory circuit 12, or may be stored in a storage medium connected to the diagnosis support apparatus 10 via the network N. In this case, the superimposed data generating function F21 realized by the processor of the processing circuitry 11 reads the trained model 11b from the memory circuit 12 and executes it, thereby generating superimposed data as diagnosis support data based on the CT image data. The trained model 11b may be constructed by an integrated circuit such as application specific integrated circuit (ASIC) or field programmable gate array (FPGA).

The accuracy of the superimposed data S′ output by the superimposed data generating function F21 may be improved by using identification data as input data that includes at least one of the appearance image (optical image) data showing the trauma, the height, the weight, and medical history of the subject, as well as the medical history of the relatives, in addition to the CT image data.

In this case, at the time of learning, the CT image data Q1, Q2, Q3, . . . , the appearance image data and the identification data of each subject are also input to the neural network Nb as the training input data. At the time of operation, the superimposed data generating function F21 inputs appearance image data and identification data of the subject to the trained model 11b read from the memory circuit 12 in addition to the CT image data Q′ of the subject, so as to output the superimposed data S′ of the subject. By using the appearance image data and the identification data of the subject in addition to the CT image data as the input data, the trained parameter data Pb′ which has been trained according to the trauma and type of the subject can be generated, and the accuracy of diagnosis can be improved as compared with the case where only CT image data is used as input data.

As described above, according to the diagnosis support apparatus 10 in the second embodiment of the diagnosis support system 1A, since the operator searches for damage while visually (or audibly) confirming the external force applied to the subject without relying on internal and external data of the vehicle or the person's fall detection data from the data acquiring system 40 (shown in FIG. 2), the efficiency can be improved as compared with the damage search that requires repeated confirmation of the injury mechanism with the subject. Further, according to the diagnosis support apparatus 10 in the second embodiment, superimposed data can be generated based on the external force indicating data of the subject and be output in the manner as shown in in FIGS. 5A to 10B, without relying on internal and external data of the vehicle or the person's fall detection data from the data acquiring system 40, which provides the operator who diagnoses the subject with effective diagnosis support data for the diagnosis. Further, according to the diagnosis support apparatus 10 in the second embodiment, superimposed data having higher accuracy can be generated based on medical image data, and the superimposed data can be easily generated since acquiring the external force indicating data at the time of diagnosis is not necessary.

### Third Embodiment

The examination data generating function F22 of the diagnosis support data generating function F2 shown in FIG. 3 will be described. The examination data generating function F22 generates examination data, which relates to at least one of the examination necessity and the examination order (imaging plan, imaging range, etc.), as the diagnosis support data based on the external force data or superimposed data acquired by the external force data acquiring function F1. Here, the external force data for generating the examination data includes at least one of the external force indicating data and the medical image data.

CT examination of “head” requires relatively higher radiation exposure in radiological examination, and it has been reported of a carcinogenic risk to the infants due to radiation exposure during head CT examination. Therefore, unnecessary head CT examination should be avoided as much as possible. Therefore, the examination data indicating the necessity of an examination such as a CT examination is generated based on the age of the patient (e.g., under 2 years old, above 2 years old and under 18 years old) who is the target of diagnosis practice, the level of consciousness of the subject, the loss of consciousness, and the mechanism of injury. In addition, the examination data generating function F22 uses the external force indicating data representing the external force applied to the subject to generate examination data showing the examination order.

In the diagnosis support system 1A shown in FIG. 11, the examination data generating function F22 shown in FIG. 3 performs a process of generating examination order based on the external force data or the superimposed data. For this process, for example, a look-up table in which the external force data or the superimposed data associated with the examination order may be used. In addition, machine learning may be used for this process. Further, deep learning using a multi-layer neural network such as CNN or CDBN may be used as the machine learning.

Here is an example showing that the examination data generating function F22 that includes a neural network Nc and generates examination order based on the external force data or the superimposed data by using the deep learning. That is, the examination data generating function F22 inputs the external force data or the superimposed data of the subject into the trained model to generate the examination data of the subject.

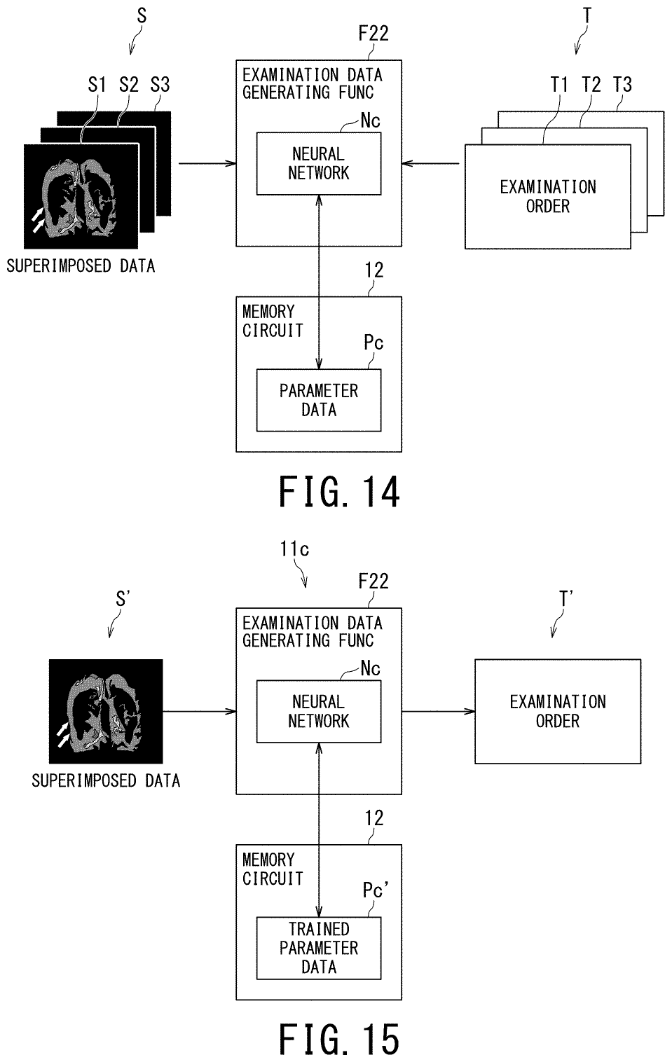

FIG. 14 is an explanatory diagram showing an example of the data flow at the time of learning. In FIGS. 14 and 15, the case where the data for generating the examination data is the superimposed data will be described. However, the superimposed data may be replaced by at least one of the external force indicating data and the medical image data.

The examination data generating function F22 sequentially updates the parameter data Pb by inputting a large number of training data and performing learning. The training data is composed of a combination of superimposed data S1, S2, S3, . . . , and examination order T1, T2, T3, . . . . The superimposed data S1, S2, S3 . . . constitutes a training input data group S. The examination order T1, T2, T3, . . . constitutes the training output data group T. The examination order T1, T2, T3, . . . may correspond to the superimposed data S1, S2, S3, . . . respectively.

The examination data generating function F22 updates the parameter data Pc such that, by the processing of neural network Nc, the superimposed data S1, S2, S3, . . . approaches the examination order T1, T2, T3, . . . each time training data is input, which is so-called learning. Generally, when the change rate of the parameter data Pc converges within the threshold value, it is determined that the learning is completed. Hereinafter, the parameter data Pc after learning is particularly referred to as learned parameter data Pc′.

It should be noted that the type of training input data and the type of input data during operation shown in FIG. 14 should be the same. For example, when the input data at the time of operation is the superimposed data including the head CT image data of the subject, the training input data group S at the time of learning should be the superimposed data including the head CT image data.

Further, the image data includes raw data generated by the image diagnostic apparatus 20 (shown in FIG. 11). That is, the input data of the neural network Nc may be raw data before scan conversion.

FIG. 15 is an explanatory diagram showing an example of data flow during operation.

At the time of operation, the examination data generating function F22 inputs the superimposed data S′ of the subject, and outputs examination order T′ of the subject using the trained parameter data Pc′.

The neural network Nc and the trained parameter data Pc′ constitute the trained model 11c. The neural network Nc is stored in the memory circuit 12 as a program. The trained parameter data Pc′ may be stored in the memory circuit 12, or may be stored in a storage medium connected to the diagnosis support apparatus 10 via the network N. In this case, the examination data generating function F22 realized by the processor of the processing circuitry 11 reads the trained model 11c from the memory circuit 12 and executes it, thereby generating examination order based on the superimposed data. The trained model 11c may be constructed by an integrated circuit such as ASIC or FPGA.

The accuracy of the examination order T′ output by the examination data generating function F22 may be improved by using identification data as input data that includes at least one of the appearance image (optical image) data showing the trauma, the height, the weight, and the medical history of the subject, as well as the medical history of the relatives, in addition to the superimposed data.

In this case, at the time of learning, the superimposed data S1, S2, S3, . . . , the appearance image data and the identification data of each subject are also input to the neural network Nc as the training input data. At the time of operation, the examination data generating function F22 inputs appearance image data and identification data of the subject to the trained model 11c read from the memory circuit 12 in addition to the superimposed data S′ of the subject so as to output the examination order T′ regarding the subject. By using the appearance image data and the identification data of the subject in addition to the superimposed data as the input data, the trained parameter data Pc′ which has been trained according to the trauma and type of the subject can be generated, and the accuracy of diagnosis can be improved as compared with the case where only superimposed data is used as input data.

As described above, according to the diagnosis support apparatus 10 in the third embodiment in the diagnosis support system 1A, in addition to the effect of the diagnosis support apparatus 10 according to the second embodiment, the examination data (examination necessity and examination order) can be generated based on the external force data or the superimposed data of the subject, which provides an operator who diagnoses a subject with effective diagnosis support data for diagnosis.

### Fourth Embodiment

The injured region data generating function F23 of the diagnosis support data generating function F2 shown in FIG. 3 will be described. The injured region data generating function F23 generates the injured region data as the diagnosis support data for identifying the injured region in the subject a based on the external force data or the superimposed data acquired by the external force data acquiring function F1. Here, the external force data for generating the injured region data includes at least one of the external force indicating data and the medical image data.

In the diagnosis support system 1A shown in FIG. 11, the injured region data generating function F23 shown in FIG. 3 performs a process of generating injured region data based on the external force data or the superimposed data. For this process, for example, a look-up table in which the external force data or the superimposed data associated with the injured region data may be used. In addition, machine learning may be used for this process. Further, deep learning using a multi-layer neural network such as CNN or CDBN may be used as the machine learning.

Here is an example showing that the injured region data generating function F23 includes a neural network Nd and generates injured region data based on the external force data or the superimposed data by using the deep learning. That is, the injured region data generating function F23 inputs the external force data or the superimposed data of the subject into the trained model to generate the injured region data of the subject.

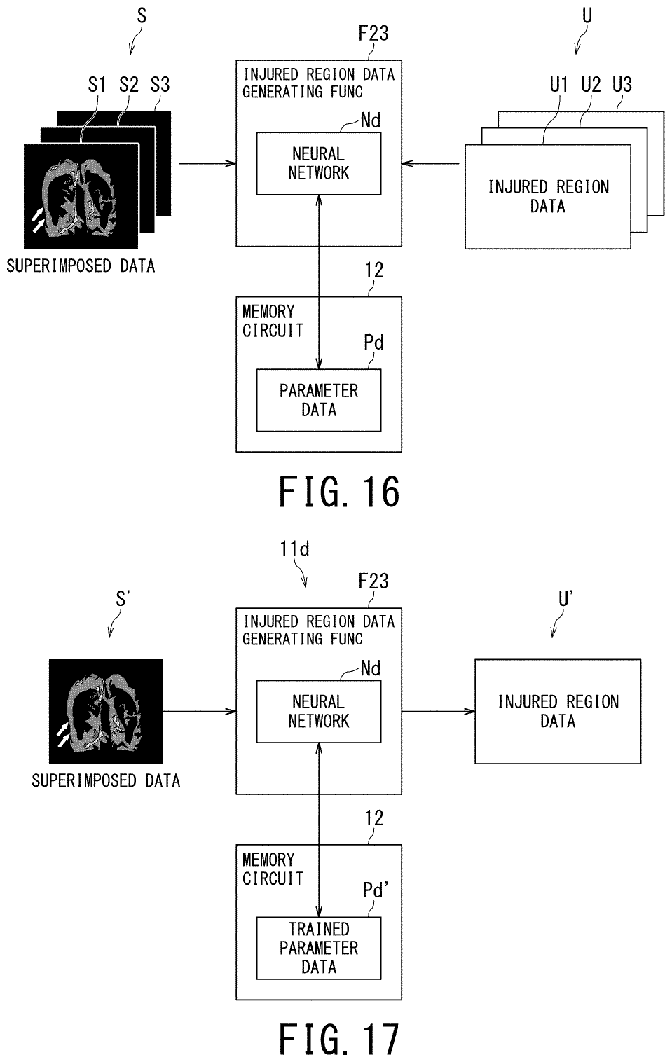

FIG. 16 is an explanatory diagram showing an example of the data flow at the time of learning. In FIGS. 16 and 17, the case where the data for generating the injured region data is the superimposed data will be described. However, the superposed data may be replaced by at least one of the external force indicating data and the medical image data. The injured region data generating function F23 sequentially updates the parameter data Pd by inputting a large number of training data and performing learning. The training data is composed of a combination of superimposed data S1, S2, S3, . . . , and injured region data U1, U2, U3, . . . . The superimposed data S1, S2, S3 . . . constitutes a training input data group S. The injured region data U1, U2, U3, . . . constitutes the training output data group U. The injured region data U1, U2, U3, . . . may correspond to the superimposed data S1, S2, S3, . . . , respectively.

The injured region data generating function F23 updates the parameter data Pd such that, by the processing of the neural network Nd, the superimposed data S1, S2, S3, . . . approaches the injured region data U1, U2, U3, . . . each time training data is input, which is so-called learning. Generally, when the change rate of the parameter data Pd converges within the threshold value, it is determined that the learning is completed. Hereinafter, the parameter data Pd after learning is particularly referred to as learned parameter data Pd′.

It should be noted that the type of training input data and the type of input data during operation shown in FIG. 16 should be the same. For example, when the input data at the time of operation is the superimposed data including the head CT image data of the subject, the training input data group S at the time of learning should be the superimposed data including the head CT image data.

Further, the image data includes raw data generated by the image diagnostic apparatus 20 (shown in FIG. 11). That is, the input data of the neural network Nd may be raw data before scan conversion.

FIG. 17 is an explanatory diagram showing an example of data flow during operation.

At the time of operation, the injured region data generating function F23 inputs the superimposed data S′ of the subject, and outputs injured region data U′ of the subject using the trained parameter data Pd′.

The neural network Nd and the trained parameter data Pd′ constitute the trained model 11d. The neural network Nd is stored in the memory circuit 12 as a program. The trained parameter data Pd′ may be stored in the memory circuit 12, or may be stored in a storage medium connected to the diagnosis support apparatus 10 via the network N. In this case, the injured region data generating function F23 realized by the processor of the processing circuitry 11 reads the trained model 11d from the memory circuit 12 and executes it, thereby generating injured region data based on the superimposed data. The trained model 11d may be constructed by an integrated circuit such as ASIC or FPGA.

The accuracy of the injured region data U′ output by the injured region data generating function F23 may be improved by using identification data as input data that includes at least one of the appearance image (optical image) data showing the trauma, the height, the weight, and the medical history of the subject, as well as the medical history of the relatives, in addition to the superimposed data.

In this case, at the time of training, the superimposed data S1, S2, S3, . . . , the appearance image data and the identification data of each subject are also input to the neural network Nd as the training input data. At the time of operation, the injured region data generating function F23 inputs appearance image data and identification data of the subject to the trained model 11d read from the memory circuit 12 in addition to the superimposed data S′ of the subject, so as to output the injured region data U′ of the subject. By using the appearance image data and the identification data of the subject in addition to the superimposed data as the input data, the trained parameter data Pd′ which has been trained according to the trauma and type of the subject can be generated, and the accuracy of diagnosis can be improved as compared with the case where only superimposed data is used as input data.

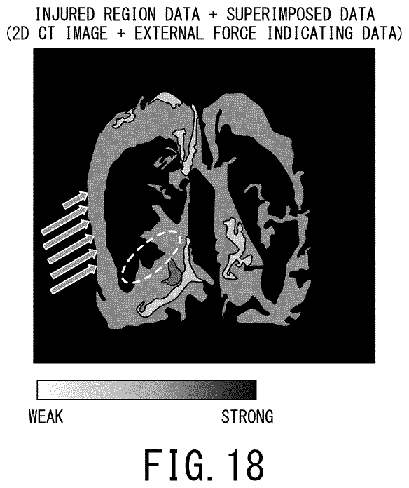

FIG. 18 is a diagram showing a display example of the injured region data in step ST4.

FIG. 18 shows superimposed data in which external force indicating data is superimposed on 3D CT image data. FIG. 18 shows an example in which injured region data (broken line) is superimposed in addition to the display of the external force indicating data shown in FIG. 5B. On the display shown in FIG. 18, the projection direction of the superimposed data to be displayed can be changed by following the operation via the input interface 13. Further, although FIG. 18 is based on the 3D CT image data, the same applies to the case based on the 2D CT image data.

According to the display of FIG. 18, by superimposing the injured region data on the CT image, the operator can perform the damage search while visually confirming the injured region data as a guide.

As described above, according to the diagnosis support apparatus 10 in the fourth embodiment in the diagnosis support system 1A, in addition to the effect of the diagnosis support apparatus 10 according to the second embodiment, the injured region data can be generated based on the external force data or the superposed data of the subject, which provides an operator who diagnoses a subject with effective diagnosis support data for diagnosis.

### Fifth Embodiment

The medical treatment data generating function F24 of the diagnosis support data generating function F2 shown in FIG. 3 will be described. The medical treatment data generating function F24 generates medical treatment data as the diagnosis support data based on the external force data or the superimposed data acquired by the external force data acquiring function F1. The medical treatment data represents the treatment plan (treatment plan, treatment/rehabilitation period) of the subject. Here, the external force data for generating the medical treatment data includes at least one of the external force indicating data and the medical image data.

In the diagnosis support system 1A shown in FIG. 11, the medical treatment data generating function F24 shown in FIG. 3 performs a process of generating medical treatment data based on the external force data or the superimposed data. For this process, for example, a look-up table in which the external force data or the superimposed data associated with the medical treatment data may be used. In addition, machine learning may be used for this process. Further, deep learning using a multi-layer neural network such as CNN or CDBN may be used as the machine learning.

Here, an example is shown in which the medical treatment data generating function F24 includes a neural network Ne and generates medical treatment data based on the external force data or the superimposed data by using the deep learning. That is, the medical treatment data generating function F24 inputs the external force data or the superimposed data of the subject into the trained model to generate the medical treatment data of the subject.

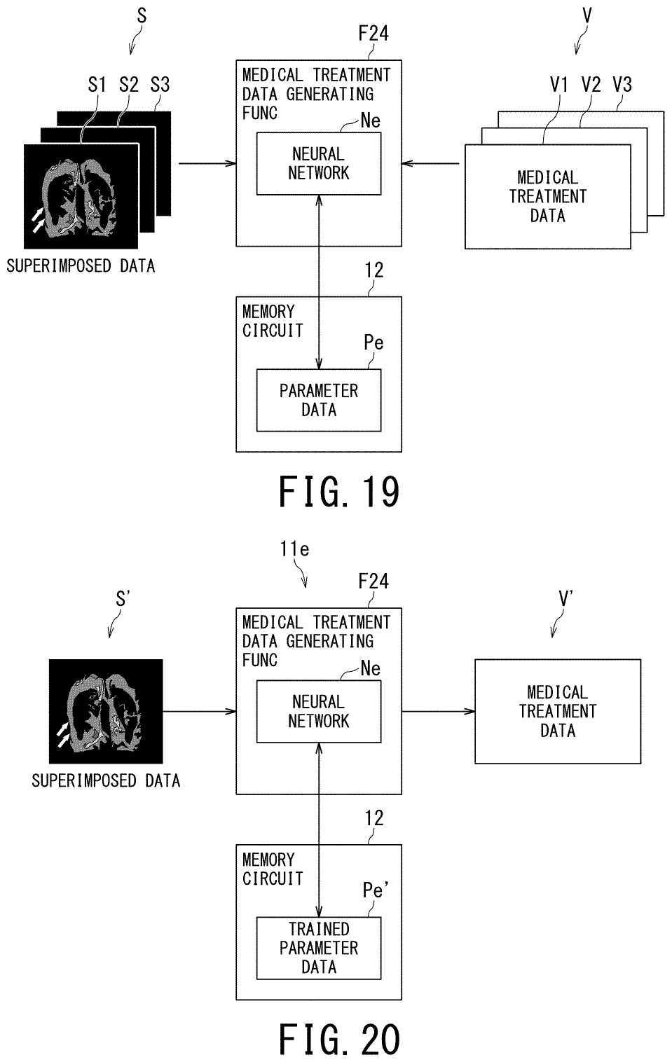

FIG. 19 is an explanatory diagram showing an example of the data flow at the time of learning. In FIGS. 19 and 20, the case where the data for generating the medical treatment data is the superimposed data will be described. However, the superimposed data may be replaced by at least one of the external force indicating data and the medical image data.

The medical treatment data generating function F24 sequentially updates the parameter data Pe by inputting a large number of training data and performing learning. The training data is composed of a combination of superimposed data S1, S2, S3, . . . , and medical treatment data V1, V2, V3, . . . . The superimposed data S1, S2, S3 . . . constitutes a training input data group S. The medical treatment data V1, V2, V3, . . . constitutes the training output data group V. The medical treatment data V1, V2, V3, . . . may correspond to the superimposed data S1, S2, S3, . . . respectively.

The medical treatment data generating function F24 updates the parameter data Pe such that, by the processing of neural network Ne, the superimposed data S1, S2, S3, . . . approaches the medical treatment data V1, V2, V3, . . . each time training data is input, which is so-called learning. Generally, when the change rate of the parameter data Pe converges within the threshold value, it is determined that the learning is completed. Hereinafter, the parameter data Pe after learning is particularly referred to as learned parameter data Pe′.

It should be noted that the type of training input data and the type of input data during operation shown in FIG. 19 should be the same. For example, when the input data at the time of operation is the superimposed data including the head CT image data of the subject, the training input data group S at the time of learning should be the superimposed data including the head CT image data.

Further, the image data includes raw data generated by the image diagnostic apparatus 20 (shown in FIG. 11). That is, the input data of the neural network Ne may be raw data before scan conversion.

FIG. 20 is an explanatory diagram showing an example of data flow during operation.

At the time of operation, the medical treatment data generating function F24 inputs the superimposed data S′ of the subject, and outputs medical treatment data V′ of the subject using the trained parameter data Pe′.

The neural network Ne and the trained parameter data Pe′ constitute the trained model 11e. The neural network Ne is stored in the memory circuit 12 as a program. The trained parameter data Pe′ may be stored in the memory circuit 12, or may be stored in a storage medium connected to the diagnosis support apparatus 10 via the network N. In this case, the medical treatment data generating function F24 realized by the processor of the processing circuitry 11 reads the trained model 11e from the memory circuit 12 and executes it, thereby generating medical treatment data based on the superimposed data. The trained model 11e may be constructed by an integrated circuit such as ASIC or FPGA.

The accuracy of the medical treatment data V′ output by the medical treatment data generating function F24 may be improved, by using identification data as input data, that includes at least one of the appearance image (optical image) data showing the trauma, the height, the weight o, and the medical history of the subject, as well as the medical history of the relatives, in addition to the superimposed data.

In this case, at the time of learning, the superimposed data S1, S2, S3, . . . , the appearance image data and the identification data of each subject are also input to the neural network Ne as the training input data. At the time of operation, the medical treatment data generating function F24 inputs appearance image data and identification data of the subject to the trained model 11e read from the memory circuit 12 in addition to the superimposed data S′ of the subject so as to output the medical treatment data V′ of the subject. By using the appearance image data and the identification data of the subject in addition to the superimposed data as the input data, the trained parameter data Pe′ which has been trained according to the trauma and type of the subject can be generated, and the accuracy of diagnosis can be improved as compared with the case where only superimposed data is used as input data.

As described above, according to the diagnosis support apparatus 10 in the fifth embodiment in the diagnosis support system 1A, in addition to the effect of the diagnosis support apparatus 10 according to the second embodiment, the medical treatment data can be generated based on the external force data or the superimposed data of the subject, which provides an operator who diagnoses a subject with effective diagnosis support data for diagnosis.

### Sixth Embodiment

Along with the progress of medical image diagnostic apparatuses such as X-ray CT apparatuses and MRI apparatuses, autopsy imaging (Ai) is used as a method for investigating the cause of death of a subject. Unlike ordinary bioimaging, it may be difficult to identify the cause of death of a deceased subject using autopsy imaging. Therefore, in order to improve the accuracy of autopsy imaging, the diagnosis support apparatus 10, as shown in FIG. 3, is provided with the cause-of-death data generating function F25.

The cause-of-death data generating function F25 generates, as the cause-of-death-identification supporting data, the cause-of-death data that shows the cause of death of the subject based on the external force data or the superimposed data acquired by the external force data acquiring function F1. Here, the external force data for generating the cause-of-death data includes at least one of the external force indicating data and the medical image data.

Here, the cause of death includes head fracture (including skull fracture, skull base fracture, etc.), brain injury (including brain contusion, diffuse axonal injury, etc.), intracerebral hemorrhage (including subdural hematoma, subarachnoid hemorrhage, etc.), cervical spine injury (including spine fractures, spinal cord injuries, etc.), visceral injury (including heart rupture, heart shaking, liver rupture, pancreas injury, etc.), fracture (including rib fracture, sternum fracture, etc.), and the like (including aortic transection, cervical dislocation, suffocation due to chest compressions, femur fracture, pelvic fracture, epidermis exfoliation, etc.).

In the diagnosis support system 1A shown in FIG. 11, the cause-of-death data generating function F25 shown in FIG. 3 performs a process of generating cause-of-death data based on the external force data or the superimposed data. For this process, for example, a look-up table in which the external force data or the superimposed data associated with the cause-of-death data are associated may be used. In addition, machine learning may be used for this process. Further, deep learning using a multi-layer neural network such as CNN or CDBN may be used as the machine learning.

Here is an example showing that the cause-of-death data generating function F25 includes a neural network Nf and generates cause-of-death data based on the external force data or the superimposed data by using the deep learning. That is, the cause-of-death data generating function F25 inputs the external force data or the superimposed data of the subject (that is, deceased subject) into the trained model to generate the cause-of-death data of the subject.

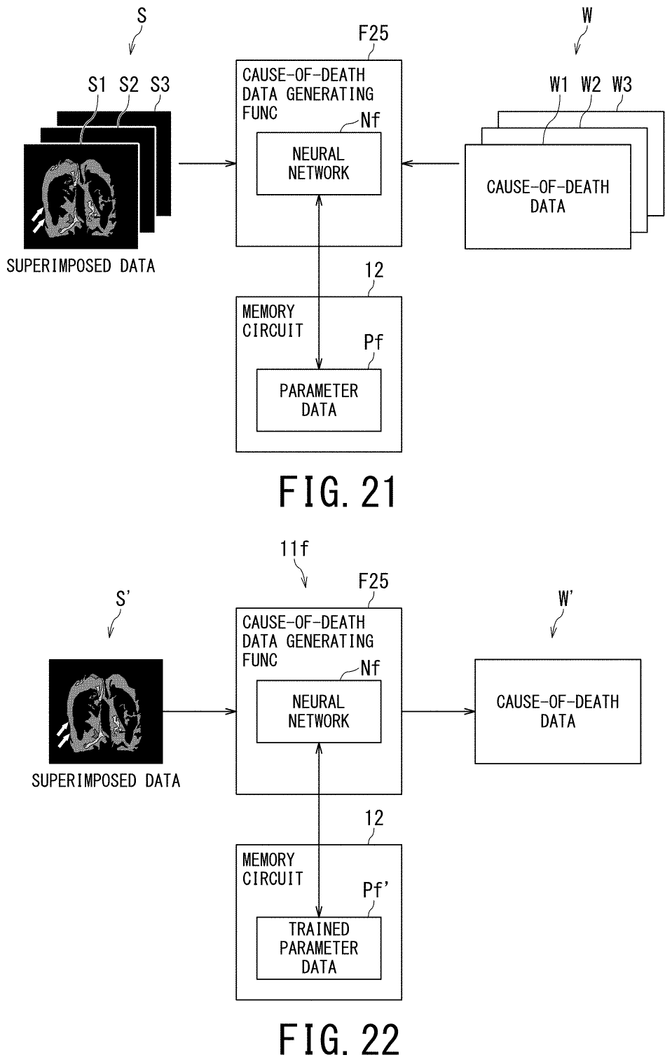

FIG. 21 is an explanatory diagram showing an example of the data flow at the time of learning. In FIGS. 21 and 22, the case where the data for generating the cause-of-death data is the superimposed data will be described. However, the superposed data may be replaced by at least one of the external force indicating data and the medical image data.

The cause-of-death data generating function F25 sequentially updates the parameter data Pf by inputting a large number of training data and performing learning. The training data is composed of a combination of superimposed data S1, S2, S3, . . . , and cause-of-death data W1, W2, W3, . . . . The superimposed data S1, S2, S3 . . . constitutes a training input data group S. The cause-of-death data W1, W2, W3, . . . constitutes the training output data group W. The cause-of-death data W1, W2, W3, . . . may correspond to the superimposed data S1, S2, S3, . . . , respectively.

The cause-of-death data generating function F25 updates the parameter data Pf such that, the by the processing of neural network Nf, the superimposed data S1, S2, S3, . . . approaches the cause-of-death data W1, W2, W3, . . . each time training data is input, which is so-called learning. Generally, when the change rate of the parameter data Pf converges within the threshold value, it is determined that the learning is completed. Hereinafter, the parameter data Pf after learning is particularly referred to as learned parameter data Pf′.

It should be noted that the type of training input data and the type of input data during operation shown in FIG. 21 should be the same. For example, when the input data at the time of operation is the superimposed data including the head CT image data of the subject, the training input data group S at the time of learning should be the superimposed data including the head CT image data.

Further, the image data includes raw data generated by the image diagnostic apparatus 20 (shown in FIG. 11). That is, the input data of the neural network Nf may be raw data before scan conversion.

FIG. 22 is an explanatory diagram showing an example of data flow during operation.

At the time of operation, the cause-of-death data generating function F25 inputs the superimposed data S′ of the subject, and outputs cause-of-death data W′ of the subject using the trained parameter data Pf′.

The neural network Nf and the trained parameter data Pf′ constitute the trained model 11f. The neural network Nf is stored in the memory circuit 12 as a program. The trained parameter data Pf′ may be stored in the memory circuit 12, or may be stored in a storage medium connected to the diagnosis support apparatus 10 via the network N. In this case, the cause-of-death data generating function F25 realized by the processor of the processing circuitry 11 reads the trained model 11f from the memory circuit 12 and executes it, thereby generating cause-of-death data based on the superimposed data. The trained model 11f may be constructed by an integrated circuit such as ASIC or FPGA.

The accuracy of the cause-of-death data W′ output by the cause-of-death data generating function F25 may be improved, by using identification data as input data, that includes at least one of the appearance image (optical image) data showing the trauma, the height, the weight, and the medical history of the subject, as well as the medical history of the relatives, in addition to the superimposed data.

In this case, at the time of learning, the superimposed data S1, S2, S3, . . . , the appearance image data and the identification data of each subject are also input to the neural network Nf as the training input data. At the time of operation, the cause-of-death data generating function F25 inputs appearance image data and identification data of the subject to the trained model 11f read from the memory circuit 12 in addition to the superimposed data S′ of the subject so as to output the cause-of-death data W′ of the subject. By using the appearance image data and the identification data of the subject in addition to the superimposed data as the input data, the trained parameter data Pf′ which has been trained according to the trauma and type of the subject can be generated, and the accuracy of cause-of-death-identification practice such as diagnosis can be improved as compared with the case where only superimposed data is used as input data.

In the above description, though the cause-of-death-identification supporting data is generated based on the external force data or the superimposed data, the cause-of-death data generating function F25 is not limited to this case. For example, the cause-of-death data generating function F25 may generate the cause-of-death data based on an image acquired during the judicial autopsy of the subject, in addition to or instead of the external force data or the superimposed data.

As described above, according to the diagnosis support apparatus 10 in the sixth embodiment, the cause-of-death data can be generated and output based on the external force data or the superimposed data of the subject, which provides the operator who identifies the cause of death of the subject with the effective diagnosis support data for autopsy imaging. Further, according to the diagnosis support apparatus 10 in the sixth embodiment, it is possible to reduce time for diagnosing the autopsy image performed by the operator who identifies the cause of death of the subject.

### Seventh Embodiment

In the above description, the superimposed data generating function F21 shown in FIG. 3 generates, as the diagnosis support data, the superimposed data in which the external force indicating data is added to the medical image data of a living subject who has received an external force. In that case, the generated superimposed data is used in biomedical imaging. However, it is not limited to that case. The superimposed data generating function F21 may generate, as the diagnosis support data, superimposed data in which external force indicating data is added to medical image data relating to a subject who has died due to external force. In that case, the generated superimposed image is used in autopsy imaging.

The superimposed data generating function F21 includes a function of generating superimposed data in which external force indicating data, as shown by symbols and/or characters, is added to medical image data as the diagnosis support data. Further, for example, the superimposed data generating function F21 generates superimposed data as an acoustic signal representing the external force indicating data. The superimposed data as the diagnosis support data in the autopsy imaging is equivalent to the superimposed data as the diagnosis support data shown in FIGS. 5A to 8 and 10A and 10B in biomedical imaging.

According to the display of FIGS. 5A to 8 and 10A and 10B, by visualizing the deformation of the skin and organs based on the external force indicating data, the operator can identify the cause of death while visually confirming the arrows and the like shown in FIG. 5A as a guide.

As described above, according to the diagnosis support apparatus 10 in the seventh embodiment of the diagnosis support system 1, by using internal and external data of the vehicle or the person's fall detection data available from the data acquiring system 40 (shown in FIG. 2), the operator can identify the cause of death while visually (or audibly) confirming the external force applied to the subject. Therefore, it is possible to improve the efficiency of identifying the cause of death of the subject whose injury mechanism cannot be confirmed.

According to at least one embodiment described above, it is possible to appropriately and efficiently support the diagnosis for subject to which an external force is applied.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. A diagnosis support apparatus comprising: processing circuitry configured to
acquire external force data regarding external force applied to a subject,
generate diagnosis support data for supporting diagnosis practice to the subject based on the acquired external force data, and
control output of the generated diagnosis support data.

2. The diagnosis support apparatus according to claim 1, wherein
the processing circuitry is configured to generate, as the diagnosis support data, superimposed data in which external force indicating data that shows the external force applied to the subject is added to medical image data, both the medical image data and the external force indicating data being included in the external force data.

3. The diagnosis support apparatus according to claim 2, wherein
the processing circuitry is configured to input the medical image data of the subject to a trained model for generating the superimposed data of the subject.

4. The diagnosis support apparatus according to claim 2, wherein
the processing circuitry is configured to control display of the generated superimposed data on a display.

5. The diagnosis support apparatus according to claim 4, wherein
the processing circuitry is configured not to display the external force indicating data in a non-display area set on an image of the medical image data, or in an area around a displayed mouse pointer.

6. The diagnosis support apparatus according to claim 1, wherein
the processing circuitry is configured to generate, as the diagnosis support data, examination data relating to at least one of an examination necessity and an examination order based on the acquired external force data.

7. The diagnosis support apparatus according to claim 6, wherein
the processing circuitry is configured to input the acquired external force data of the subject to a trained model for generating the examination order of the subject among the examination data.

8. The diagnosis support apparatus according to claim 2, wherein
the processing circuitry is configured to generate, as the diagnosis support data, examination data relating to at least one of an examination necessity and an examination order based on the generated superimposed data.

9. The diagnosis support apparatus according to claim 8, wherein
the processing circuitry is configured to input the superimposed data of the subject to a trained model for generating the examination order of the subject among the examination data based on the generated superimposed data.

10. The diagnosis support apparatus according to claim 1, wherein
the processing circuitry is configured to generate, as the diagnosis support data, injured region data identifying an injured region of the subject based on the acquired external force data.

11. The diagnosis support apparatus according to claim 10, wherein
the processing circuitry is configured to input the external force data of the subject to a trained model for generating the injured region data of the subject based on the acquired external force data.

12. The diagnosis support apparatus according to claim 2, wherein
the processing circuitry is configured to generate, as the diagnosis support data, injured region data identifying an injured region of the subject based on the generated superimposed data.

13. The diagnosis support apparatus according to claim 12, wherein
the processing circuitry is configured to input the superimposed data of the subject to a trained model for generating the injured region data of the subject based on the generated superimposed data.

14. The diagnosis support apparatus according to claim 1, wherein
the processing circuitry is configured to generate, as the diagnosis support data, medical treatment data representing a medical treatment plan of the subject based on the acquired external force data.

15. The diagnosis support apparatus according to claim 14, wherein
the processing circuitry is configured to input the acquired external force data of the subject to a trained model for generating the medical treatment data of the subject.

16. The diagnosis support apparatus according to claim 2, wherein
the processing circuitry is configured to generate, as the diagnosis support data, medical treatment data representing a medical treatment plan of the subject based on the generated superimposed data.

17. The diagnosis support apparatus according to claim 16, wherein
the processing circuitry is configured to input the generated superimposed data of the subject to a trained model for generating the medical treatment data of the subject.

18. The diagnosis support apparatus according to claim 2, wherein
the processing circuitry is configured to generate, as the diagnosis support data, cause-of death data representing a cause of death of the subject based on the generated superimposed data.

19. The diagnosis support apparatus according to claim 18, wherein
the processing circuitry is configured to input the generated superimposed data of the subject to a trained model for generating the cause-of-death data of the subject.

20. A method for supporting diagnosis comprising: steps of
acquiring external force data regarding external force applied to a subject,
generating diagnosis support data for supporting diagnosis practice to the subject based on the acquired external force data, and
controlling output of the generated diagnosis support data.
